SEQUENCE LISTING (37 sequences)

SEQ ID NO: 1 - protein, 18 aa, Artificial Sequence
  HA2 fusion peptide
GLFGAIAGFIENGWEGMI

SEQ ID NO: 2 - protein, 4 aa, Artificial Sequence
  Linker
KKKK

SEQ ID NO: 3 - protein, 5 aa, Artificial Sequence
  Linker
KKKKK

SEQ ID NO: 4 - protein, 4 aa, Artificial Sequence
  Linker
RRRR

SEQ ID NO: 5 - protein, 5 aa, Artificial Sequence
  Linker
RRRRR

SEQ ID NO: 6 - protein, 4 aa, Artificial Sequence
  Linker
KKRK

SEQ ID NO: 7 - protein, 4 aa, Artificial Sequence
  Linker
RRKR

SEQ ID NO: 8 - protein, 4 aa, Artificial Sequence
  Linker
RKKR

SEQ ID NO: 9 - protein, 4 aa, Artificial Sequence
  Linker
KRRK

SEQ ID NO: 10 - protein, 4 aa, Artificial Sequence
  Linker
CKKK

SEQ ID NO: 11 - protein, 5 aa, Artificial Sequence
  Linker
CKKKK

SEQ ID NO: 12 - protein, 6 aa, Artificial Sequence
  Linker
CKKKKK

SEQ ID NO: 13 - protein, 4 aa, Artificial Sequence
  Linker
CRRR

SEQ ID NO: 14 - protein, 5 aa, Artificial Sequence
  Linker
CRRRR

SEQ ID NO: 15 - protein, 6 aa, Artificial Sequence
  Linker
CRRRRR

SEQ ID NO: 16 - protein, 4 aa, Artificial Sequence
  Linker
CRKR

SEQ ID NO: 17 - protein, 4 aa, Artificial Sequence
  Linker
CKRK

SEQ ID NO: 18 - protein, 5 aa, Artificial Sequence
  Linker
CKKRK

SEQ ID NO: 19 - protein, 5 aa, Artificial Sequence
  Linker
CRRKR

SEQ ID NO: 20 - protein, 5 aa, Artificial Sequence
  Linker
CRKKR

SEQ ID NO: 21 - protein, 5 aa, Artificial Sequence
  Linker
CKRRK

SEQ ID NO: 22 - protein, 4 aa, Artificial Sequence
  Linker
KKKC

SEQ ID NO: 23 - protein, 5 aa, Artificial Sequence
  Linker
KKKKC

SEQ ID NO: 24 - protein, 6 aa, Artificial Sequence
  Linker
KKKKKC

SEQ ID NO: 25 - protein, 4 aa, Artificial Sequence
  Linker
RRRC

SEQ ID NO: 26 - protein, 5 aa, Artificial Sequence
  Linker
RRRRC

SEQ ID NO: 27 - protein, 6 aa, Artificial Sequence
  Linker
RRRRRC

SEQ ID NO: 28 - protein, 4 aa, Artificial Sequence
  Linker
RKRC

SEQ ID NO: 29 - protein, 4 aa, Artificial Sequence
  Linker
KRKC

SEQ ID NO: 30 - protein, 5 aa, Artificial Sequence
  Linker
KKRKC

SEQ ID NO: 31 - protein, 5 aa, Artificial Sequence
  Linker
RRKRC

SEQ ID NO: 32 - protein, 5 aa, Artificial Sequence
  Linker
RKKRC

SEQ ID NO: 33 - protein, 5 aa, Artificial Sequence
  Linker
KRRKC

SEQ ID NO: 34 - protein, 15 aa, Artificial Sequence
  HA2 fusion peptide
RKKRGLFGAIAGFIE

SEQ ID NO: 35 - protein, 12 aa, Artificial Sequence
  HA2 fusion peptide
GLFGAIAGFKKC

SEQ ID NO: 36 - protein, 22 aa, Artificial Sequence
  HA2 fusion peptide
GLFGAIAGFIENGWEGMIKKKC

SEQ ID NO: 37 - protein, 16 aa, Artificial Sequence
  HA2 fusion peptide
RKKRGLFGAIAGFKKC

(19) United States
(12) Patent Application Publication
(10) Pub. No.: US 2014/0234360 A1
(43) Pub. Date: Aug. 21, 2014

(54) INFLUENZA VACCINE

(71) Applicant: The United States of America, as represented by the Secretary, Department of Health and Human Serv, Bethesda, MD (US)

(72) Inventors: Joanna Kubler-Kielb, Bethesda, MD (US); Jerry Keith, Olney, MD (US); Rachel Schneerson, Bethesda, MD (US); Zuzana Biesova, Rockville, MD (US)

(73) Assignee: The United States of America, as represented by the Secretary, Dept.of Health and Human Services, Bethesda, MD (US)

(21) Appl. No.: 14/348,321

(22) PCT Filed: Sep. 28, 2012
(86) PCT No.: PCT/US2012/057863

§ 371 Date: Mar. 28, 2014

Related U.S. Application Data
(60) Provisional application No. 61/541,942, filed on Sep. 30, 2011.

(51) Int. Cl. A61K 47/48

(52) U.S. Cl. CPC A61K 47/4833; A61K 47/48261
USPC 424/186.1; 530/363; 424/196.11

(57) ABSTRACT

Disclosed are immunogenic conjugates having the general formula: HA2-XXX-Pr, where HA2 is the influenza HA2 fusion peptide or a portion thereof, XXX is a linker and Pr is the carrier. Methods of producing an immune response in a subject using the disclosed immunogenic conjugates, as well as methods of treating, ameliorating or preventing influenza infection, are also disclosed.

### RELATED APPLICATION

This application claims the benefit of U.S. Provisional Application No. 61/541,942, filed Sep. 30, 2011, which is incorporated by reference in its entirety.

### FIELD

Disclosed herein are immunogens and immunogenic compositions produced from influenza hemagglutinin 2 (HA2) derived peptides and methods of treating subjects with such immunogens.

### BACKGROUND

Influenza virus types A and B are members of the orthomyxoviridae family of viruses that cause influenza infection. Influenza A and B viruses primarily infect the nasopharyngeal and oropharyngeal cavities and produce highly contagious, acute respiratory disease that can result in significant morbidity and high economic costs.

Typical influenza viral infections in humans have a relatively short incubation period of 1 to 2 days, with symptoms that last about a week including an abrupt onset of fever, sore throat, cough, headache, myalgia, and malaise. However, when a subject is infected with a highly virulent strain of influenza these symptoms can progress rapidly to pneumonia and in some circumstances death.

Genetic reassortment between human and avian influenza viruses can result in a virus with a novel hemagglutinin (HA) of avian origin, against which humans lack immunity. Recombination between avian strains and human strains in coinfected individuals has given rise to recombinant influenza viruses to which immunity is lacking in the human population, resulting in influenza pandemics. In the 20^{th} century, the pandemics of 1918, 1957 and 1968 were the result of such antigenic shifts. The threat of pandemic outbreaks of highly virulent influenza presents a serious risk to human and animal health worldwide.

Due to the potential lethality of these influenza strains, there remains a need to develop vaccines that are protective against infection by influenza strains of human and non-human populations.

### SUMMARY OF THE DISCLOSURE

The present disclosure provides novel compositions and influenza vaccines for vaccinating humans, non-human mammals and avian populations against human, avian, and/or pandemic strains of influenza virus and overcoming the poor immunogenicity, lack of universal immune response against influenza virus strains, and manufacturing drawbacks of currently available influenza vaccines, which have been adapted to elicit an immune response against human and avian strains of influenza.

Disclosed herein are immunogenic conjugates that include an influenza hemagglutinin 2 (HA2) fusion peptide covalently linked to a carrier protein. The conjugates have the general formula:

where HA2 is the influenza HA2 fusion peptide, XXX is a linker and Pr is the carrier.

In some examples, the HA2 fusion peptide and the carrier protein are linked by a linker between a lysine amino acid residue present on the carrier protein and a cysteine amino acid residue present on the HA2 fusion peptide and the conjugates have the formula:

HA2-Cys-S—XXX—NH-Lys-Pr,

where HA2 is the influenza HA2 fusion peptide, XXX is a linker; Cys is a cysteine amino acid residue; S is the sulfur present in the cysteine amino acid residue; NH is the amine group present in a lysine residue; Lys is a lysine amino acid residue and Pr is the carrier protein.

In some examples, the conjugates of the HA2 fusion peptide and the carrier protein are linked by a thioether linkage between a lysine amino acid residue on the carrier protein and a cysteine amino acid residue present on the HA2 fusion peptide and have the formula:

HA2-Cys-S—CH₂—C(O)—NH—CH₂—CH₂—C(O)—NH-Lys-Pr,

where HA2 is the influenza HA2 fusion peptide; Cys is a cysteine amino acid residue; S is the sulfur present in the cysteine amino acid residue; CH₂—CO—NH—CH₂—CH₂—CO is the linking group; NH is the amine group present in a lysine residue; Lys is a lysine amino acid residue and Pr is the carrier protein.

In some embodiments, the HA2 fusion peptide includes one or more polybasic amino acid sequences, for example at the N-terminus, C-terminus or both. In some embodiments the HA2 fusion peptide includes a cysteine residue for example at the N- or C-terminus.

In some embodiments, the carrier is linked to the C-terminal end of the HA2 fusion peptide. In some embodiments, the carrier is linked to the N-terminus of the HA2 fusion peptide.

The immunogens disclosed herein are useful in the context of immunogenic compositions, including vaccines.

The present disclosure also provides methods for eliciting or producing an immune response against influenza. The methods disclosed herein involve administering one or more of the disclosed immunogenic conjugates, immunogens and/or immunogenic compositions to a subject. Administration of the immunogenic conjugates, immunogens and/or immunogenic compositions can elicit an immune response that protects the subject from serious disease or death due to infection by influenza. Typically, the immune response includes neutralizing antibodies that bind to at least one influenza antigen, such as an HA antigen.

The foregoing and other features, and advantages of this disclosure will become more apparent from the following detailed description, which proceeds with reference to the accompanying figures.

### BRIEF DESCRIPTION OF THE DRAWINGS

FIG. 1 is a schematic representation of the conjugation of a HA2 fusion peptide to a carrier protein via a thioether linkage.

FIG. 2 is a digital image of the results of polyacrylamide gel electrophoresis.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. The Sequence Listing is submitted as an ASCII text file in the form of the file named Sequence.txt (˜8 kb), which was created on Sep. 26 27, 2012, and is incorporated by reference herein. In the accompanying Sequence Listing:

SEQ ID NOs: 1-10, 43-46 are the amino acid sequences of exemplary HA2 fusion peptides.

SEQ ID NOs: 11-42 are the amino acid sequences of exemplary polybasic sequences.

### DETAILED DESCRIPTION

### I. Introduction

Influenza A virus causes annual epidemics of acute respiratory disease and worldwide pandemics. The lipid envelope surface of the virus is covered with approximately 1000 protruding protein spikes each including three identical hemagglutinin (HA) molecules. Serum IgG against these spikes (including HA antibodies) neutralizes viral infectivity.

The segmented viral genome of influenza is negative, single-stranded RNA. During replication of the viral genome by the virus encoded error-prone RNA polymerase, mutations are introduced into the replicating virus. Those mutations which are beneficial to viral survival, such as eluding the host immune defenses, are conserved by the evolving virus. This so called genetic drift in the gene encoding the HA protein necessitate frequent changes of vaccines in order to be effective against the rapidly evolving viruses in the population.

Although uncommon, a major genetic shift can occur by reassortment of the segmented RNA genome leading to a change in the circulating sub-type in humans, potentially causing a pandemic. Currently, 16 sub-types of HA have been identified in avian influenza A viruses, three of which are also identified in human strains.

The speed of vaccine production, especially for potentially pandemic viruses, is limited by current manufacturing processes. Each year, circulating virus strains are characterized by RNA sequence and immunological cross-reactivity with other strains. From these data, candidate strains are identified and released to vaccine manufacturers. The majority of influenza manufacturing relies on embryonated chicken eggs to propagate the vaccine virus. If the circulating virus strain is not lethal to eggs, it is grown in eggs, purified, inactivated chemically, tested and then distributed. Highly pathogenic strains that cannot be grown in eggs require genetic modification, which prolongs the production schedule. Typically, the current vaccine production process produces only enough vaccine for one dose per person and is not available until just before the flu season begins in the fall. To put this number in perspective, the peak of the 1918 influenza pandemic lasted only four months and resulted in 50 to 100 million deaths. Thus, innovative vaccines that combat the diversity in influenza strains are urgently needed. The current disclosure meets those needs by providing immunogenic compositions, such as vaccines, that are useful in the treatment and/or inhibition of influenza infection from multiple influenza strains and subtypes.

A fusion peptide that includes the 9 to 20 amino acids at the N-terminus of the influenza HA2 protein is highly conserved among all A and B influenza viruses. Monoclonal antibodies against this peptide are capable of binding all influenza viruses HA proteins and inhibit viral growth by impeding the fusion process between the virus and the target cell.

As disclosed herein, the inventors have prepared protein-conjugates able to induce antibodies to this fusion peptide, which is located immediately adjacent to the protease cleavage site that separates the HAO protein into its HA1 and HA2 components. Cleavage at this site releases the N-terminal end of the HA2 fusion domain from HA1, which is essential for influenza virus infectivity and replication. The disclosed protein-conjugates therefore provide a broadly cross-reactive influenza vaccine that can treat and/or inhibit influenza infection from multiple strains, and potentially eliminate the need for yearly vaccine treatments with standard influenza vaccines.

### II. Summary of Terms

Unless otherwise noted, technical terms are used according to conventional usage. Definitions of common terms in molecular biology may be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710).

The singular terms “a,” “an,” and “the” include plural referents unless context clearly indicates otherwise. Similarly, the word “or” is intended to include “and” unless the context clearly indicates otherwise. The term “comprises” means “includes.” In case of conflict, the present specification, including explanations of terms, will control.

To facilitate review of the various embodiments of this disclosure, the following explanations of terms are provided:

Adaptive immune response: A response of a cell of the immune system, such as a B cell, T cell, to a stimulus. In some cases, an immune response is a T cell response, such as a CD4+ response or a CD8+ response. Alternatively, the response is a B cell response, and results in the production of specific antibodies. A “protective immune response” is an immune response that inhibits a detrimental function or activity of a pathogen such as an influenza virus, reduces infection by a pathogenic influenza virus, or decreases symptoms (including death) that result from infection by the pathogenic organism. A protective immune response can be measured, for example, by the inhibition of viral replication or plaque formation in a plaque reduction assay or ELISA-neutralization assay (NELISA), or by measuring resistance to viral challenge in vivo. A cell-mediated immune response can be measured by various immunological assays, e.g., ELISpot, tetramer-labeling or cytotoxicity assay.

Adjuvant: A substance that non-specifically enhances the immune response to an immunogen, such as the immunogens disclosed herein. Adjuvants include a suspension of minerals (alum, aluminum hydroxide, or phosphate) on which antigen is adsorbed; or water-in-oil emulsion in which immunogen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages) Immunostimulatory oligonucleotides (such as those including a CpG motif) can also be used as adjuvants (for example see U.S. Pat. No. 6,194,388; U.S. Pat. No. 6,207,646; U.S. Pat. No. 6,214,806; U.S. Pat. No. 6,218,371; U.S. Pat. No. 6,239,116; U.S. Pat. No. 6,339,068; U.S. Pat. No. 6,406,705; and U.S. Pat. No. 6,429,199). Adjuvants include biological molecules (a “biological adjuvant”), such as costimulatory molecules. Development of vaccine adjuvants for use in humans is reviewed in Singh et al. (Nat. Biotechnol. 17:1075-1081, 1999), which discloses aluminum salts, such as aluminum hydroxide (ALHYDROGELO, available from Brenntag Biosector, Copenhagen, Denmark and AMPHOGELO, Wyeth Laboratories, Madison, N.J.) and MF59 microemulsion.

Administration: The introduction of a composition into a subject by a chosen route. Administration can be local or systemic. All methods of administration are contemplated by this disclosure.

Amplification: Increasing the number of copies of a nucleic acid molecule. The resulting amplification products are called “amplicons.” Amplification of a nucleic acid molecule (such as a DNA or RNA molecule) refers to use of a technique that increases the number of copies of a nucleic acid molecule in a sample, for example the number of copies of an influenza HA2 nucleic acid. An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

Other examples of in vitro amplification techniques include quantitative real-time PCR; reverse transcriptase PCR (RT-PCR); real-time PCR (rt PCR); real-time reverse transcriptase PCR (rt RT-PCR); nested PCR; strand displacement amplification (see U.S. Pat. No. 5,744,311); transcription-free isothermal amplification (see U.S. Pat. No. 6,033,881, repair chain reaction amplification (see WO 90/01069); ligase chain reaction amplification (see European patent publication EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134) amongst others.

Analog, Derivative or Mimetic: An analog is a molecule that differs in chemical structure from a parent compound, for example a homolog (differing by an increment in the chemical structure, such as a difference in the length of an alkyl chain), a molecular fragment, a structure that differs by one or more functional groups, a change in ionization. Structural analogs are often found using quantitative structure activity relationships (QSAR), with techniques such as those disclosed in Remington (The Science and Practice of Pharmacology, 19th Edition (1995), chapter 28). A derivative is a biologically active molecule derived from the base structure. A mimetic is a molecule that mimics the activity of another molecule, such as a biologically active molecule. Biologically active molecules can include chemical structures that mimic the biological activities of a compound.

Animal: Living multi-cellular vertebrate organisms, a category that includes, for example, mammals and birds. The term mammal includes both human and non-human mammals. Similarly, the term “subject” includes both human and non-human subjects, including birds and non-human mammals, such as non-human primates, companion animals (such as dogs and cats), livestock (such as pigs, sheep, cows), as well as non-domesticated animals. The term subject applies regardless of the stage in the organism's life-cycle. Thus, the term subject applies to an organism in utero or in ovo, depending on the organism (that is, whether the organism is a mammal or a bird, such as a domesticated or wild fowl).

Antibody: A protein (or protein complex) that includes one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. In some examples, an antibody is one that specifically binds a HA2 fusion peptide.

Antigen: A compound, composition, or substance that can stimulate the production of antibodies or a T cell response in an animal, including compositions that are injected or absorbed into an animal. An antigen reacts with the products of specific humoral or cellular immunity. Examples of antigens include, but are not limited to, polypeptides, peptides, lipids, polysaccharides, combinations thereof (such as glycopeptides) and nucleic acids containing antigenic determinants, such as those recognized by an immune cell. In some examples, antigens include peptides derived from a pathogen of interest, such as influenza. In specific examples, an antigen is derived from influenza, such as an antigen including a HA2 fusion protein. The term “antigen” includes all related antigenic epitopes. An “antigenic polypeptide” is a polypeptide to which an immune response, such as a T cell response or an antibody response, can be stimulated.

cDNA (complementary DNA): A piece of DNA lacking internal, non-coding segments (introns) and regulatory sequences that determine transcription. cDNA is typically synthesized in the laboratory by reverse transcription from RNA, such as messenger RNA extracted from cells and/or viral RNA, for exam RNA encoding an influenza HA2 polypeptide. In the context of preparing immunogen, immunogenic conjugates and vaccines of the present disclosure, for example from a polynucleotide sequences that encode influenza antigen (such as HA), a cDNA can be prepared, for example by reverse transcription or amplification (e.g., by the polymerase chain reaction, PCR) from a negative stranded influenza RNA genome (or genome segment).

Carrier: An immunogenic molecule to which an antigen, such as an influenza antigen (for example HA), can be bound. When bound to a carrier, the bound molecule may become more immunogenic. Carriers are chosen to increase the immunogenicity of the bound molecule which are diagnostically, analytically, and/or therapeutically beneficial. In some examples, a carrier is chosen such that multiple copies of an antigen can be linked to the carrier, for example covalently linked. Covalent linking of a molecule to a carrier confers enhanced immunogenicity and T-cell dependence (Pozsgay et al., PNAS 96:5194-97, 1999; Lee et al., J. Immunol. 116:1711-18, 1976; Dintzis et al., PNAS 73:3671-75, 1976). Useful carriers include polymeric carriers, which can be natural (for example, proteins from bacteria or viruses), semi-synthetic or synthetic materials containing one or more functional groups to which a reactant moiety can be attached.

Examples of bacterial products for use as carriers include bacterial toxins, such as diphtheria toxoid (including recombinant toxins that have been genetically detoxified and analogs or derivatives capable of eliciting an immune response against an biological molecule covalently linked to the carrier protein), LF, EF and LeTx (a heterodimer of protective antigen and lethal factor), and other bacterial toxins and toxoids, such as tetanus toxoid, P. aeruginosa exotoxin/toxoid, pertussis toxin/toxoid, and C. perfringens exotoxin/toxoid. Other examples of carriers include keyhole limpet hemocyanin, horseshoe crab hemocyanin, edestin, mammalian serum albumins, and mammalian immunoglobulins, Viral proteins, such as hepatitis B surface antigen and core antigen can also be used as carriers.

Conjugate: A composition composed of two heterologous molecules (such as an influenza antigen and a carrier, such as a protein carrier) linked together useful for stimulating or eliciting a specific immune response in a vertebrate. In some embodiments, the immune response is protective in that it enables the vertebrate animal to better resist infection from the organism against which the immunogenic conjugate is directed. One specific example of a type of immunogenic conjugate is a vaccine, such as a conjugate vaccine.

Contacting: Placement in direct physical association; includes both in solid and liquid form. Contacting includes contact between one molecule and another molecule, for example the amino acid on the surface of one polypeptide, such as an antigen, that contact another polypeptide, such as an antibody. Contacting also includes administration, such as administration of a disclosed antigen to a subject by a chosen route.

Control: A reference standard. In some embodiments, the control is a negative control sample obtained from a healthy patient. In other embodiments, the control is a positive control sample obtained from a patient diagnosed with influenza infection. In still other embodiments, the control is a historical control or standard reference value or range of values (such as a previously tested control sample, such as a group of influenza patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Covalent bond: An interatomic bond between two atoms, characterized by the sharing of one or more pairs of electrons by the atoms. The terms “covalently bound” or “covalently linked” refer to making two separate molecules into one contiguous molecule. The terms include reference to joining an antigen (such as an influenza antigen) either directly or indirectly to a carrier molecule, for example indirectly with an intervening linker molecule, such as a peptide or non-peptide linker.

Effective amount or therapeutically effective amount: A quantity of a specified agent sufficient to achieve a desired effect in a subject being treated with that agent. For example, this may be the amount of a vaccine and/or immunogenic conjugate disclosed herein useful in increasing resistance to, preventing, ameliorating, and/or treating infection and disease caused by influenza virus infection in a subject. A therapeutically effective amount of an agent is an amount sufficient to increase resistance to, prevent, ameliorate, and/or treat infection and disease caused by influenza virus infection in a subject without causing a substantial cytotoxic effect in the subject. The effective amount of an agent useful for increasing resistance to, preventing, ameliorating, and/or treating infection and disease caused by influenza virus infection in a subject will be dependent on the subject being treated, the severity of the affliction, and the manner of administration of the therapeutic composition.

Epitope or Antigenic epitope: An antigenic determinant. These are particular chemical groups, contiguous or non-contiguous peptide or saccharide sequences on a molecule that are antigenic, that is, they bind to a specific Ab. As part of a large molecule they may be immunogenic. An antibody binds a particular antigenic epitope based on the three dimensional structure of the antibody and the matching (or cognate) epitope. “Epitope” or “antigenic determinant” refers to a site of an antigen to which B and/or T cells respond. Epitopes can be formed both from contiguous amino acids or noncontiguous amino acids juxtaposed by tertiary folding of an antigenic polypeptide. Methods of determining spatial conformation of epitopes include, for example, x-ray crystallography and multi-dimensional nuclear magnetic resonance spectroscopy.

Expression: Translation of a nucleic acid into a protein, for example the translation of a nucleic acid molecule encoding an immunogenic fragment of an influenza HA2 fusion polypeptide into a peptide.

Expression control sequences: Nucleic acid sequences that regulate the expression of a heterologous nucleic acid sequence to which it is operatively linked, for example the expression of nucleic acid encoding an immunogenic fragment of an influenza HA2 fusion polypeptide operably linked to expression control sequences. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (such as metallothionein promoter) or from mammalian viruses (such as the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

A polynucleotide can be inserted into an expression vector that contains a promoter sequence, which facilitates the efficient transcription of the inserted genetic sequence of the host. The expression vector typically contains an origin of replication, a promoter, as well as specific nucleic acid sequences that allow phenotypic selection of the transformed cells.

Host cells: Cells in which a polynucleotide, for example, a polynucleotide vector, can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used. Thus, vectors encoding the peptides of the vaccines described herein can be introduced into host cells where their polynucleotide sequences (including those encoding influenza antigen(s)) can be expressed, for example to produce recombinant influenza antigens and/or carriers.

Immunogen: A compound, composition, or substance that can stimulate the production of antibodies or a T cell response in an animal, including compositions that are injected, absorbed or otherwise introduced into an animal. Administration of an immunogen can lead to protective immunity and/or proactive immunity against a pathogen of interest. An influenza immunogen can include hemagglutinin (HA) or a portion or fragment thereof, such as a portion of an influenza HA2 fusion peptide.

Immune response: A response of a cell of the immune system, such as a B cell, T cell, or monocyte, to a stimulus. In one embodiment, the response is specific for a particular antigen (an “antigen-specific response”). In one embodiment, an immune response is a T cell response, such as a CD4+ response or a CD8+ response. In another embodiment, the response is a B cell response, and results in the production of specific antibodies.

Influenza virus: Influenza viruses are enveloped negative-sense viruses belonging to the orthomyxoviridae family. Influenza viruses are classified on the basis of their core proteins into three distinct types: A, B, and C. Within these broad classifications, subtypes are further divided based on the characterization of two antigenic surface proteins hemagglutinin (HA or H) and neuraminidase (NA or N). While B and C type influenza viruses are largely restricted to humans, influenza A viruses are pathogens of a wide variety of species including humans, non-human mammals, and birds. Periodically, non-human strains, particularly of avian influenza, have infected human populations, in some cases causing severe disease with high mortality. Recombination between such avian and non-human mammal strains with human strains in coinfected individuals has given rise to recombinant influenza viruses to which immunity is lacking in the human population, resulting in influenza pandemics. Three such pandemics occurred during the twentieth century (pandemics of 1918, 1957, and 1968) and resulted in numerous deaths world-wide.

Influenza viruses have a segmented single-stranded (negative or antisense) genome. The influenza virion consists of an internal ribonucleoprotein core containing the single-stranded RNA genome and an outer lipoprotein envelope lined by a matrix protein. The segmented genome of influenza consists of eight linear RNA molecules that encode ten polypeptides. Two of the polypeptides, HA and NA include the primary antigenic determinants or epitopes required for a protective immune response against influenza. Based on the antigenic characteristics of the HA and NA proteins, influenza strains are classified into subtypes. For example, recent outbreaks of avian influenza in Asia have been categorized as H5N1, H7N7, and H9N2 based on their HA and NA phenotypes.

HA is a surface glycoprotein which projects from the lipoprotein envelope and mediates attachment to and entry into cells. The HA protein is approximately 566 amino acids in length, and is encoded by an approximately 1780 base polynucleotide sequence of segment 4 of the genome. Polynucleotide and amino acid sequences of HA (and other influenza antigens) isolated from recent, as well as historic, influenza strains can be found, for example in the GENBANK® database (available on the world wide web at ncbi.nlm.nih.gov/entrez) or the Influenza Sequence Database of Los Alamos National Laboratories (LANL) (available on the world wide web at flu.lanl.gov). For example, recent H1 subtype HA sequences include: AY038014, and J02144; recent H3 subtype HA sequences include: AY531037, M29257, and U97740; H5 subtype HA sequences include: AY075033, AY075030, AY818135, AF046097, AF046096, and AF046088; recent H7 subtype HA sequences include: AJ704813, AJ704812, and Z47199; and, recent H9 subtype HA sequences include: AY862606, AY743216, and AY664675.

The mature influenza HAO polypeptide includes a basic amino acid cleavage site between the HA1 and HA2 domains, and a C-terminal transmembrane domain. In highly pathogenic strains of the virus, the basic amino acid cleavage site is replaced with polybasic amino acids. The N-terminal leader amino acid sequence of the HA1 domain is cleaved during processing.

Immunogenic composition: A composition useful for stimulating or eliciting a specific immune response in a vertebrate. In some embodiments, the immunogenic response is protective in that it enables the vertebrate animal to better resist infection or disease progression from the organism against which the immunogenic composition is directed. One specific example of a type of immunogenic composition is a vaccine.

Inhibiting or treating a disease: Inhibiting the full development of a disease or condition, for example, in a subject who is at risk for a disease such as an influenza viral infection. For example, inhibiting an influenza infection refers to decreasing symptoms resulting from infection by the virus, such as preventing the development of symptoms in a person who is known to have been exposed to the virus or to lessening virus number or infectivity of a virus in a subject exposed to the virus. “Treatment” refers to a therapeutic intervention that ameliorates a sign or symptom of a disease or pathological condition after it has begun to develop. The term “ameliorating,” with reference to a disease, pathological condition or symptom refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease.

Isolated: An “isolated” biological component (such as a nucleic acid molecule, protein or peptide) has been substantially separated or purified away from other biological components in the cell of the organism in which the component naturally occurs, such as other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant expression in a host cell, as well as chemically synthesized nucleic acids or proteins, or fragments thereof.

Linker: A compound or moiety that acts as a molecular bridge to operably link two different molecules, wherein one portion of the linker is operably linked to a first molecule and wherein another portion of the linker is operably linked to a second molecule. The two different molecules can be linked to the linker in a step-wise manner. There are no particular size or content limitations for the linker so long as it can fulfill its purpose as a molecular bridge. Linkers are known to those skilled in the art to include, but are not limited to, chemical chains, chemical compounds, carbohydrate chains, peptides, haptens and the like. The linkers can include, but are not limited to, homobifunctional linkers and hetero-bifunctional linkers. Hetero-bifunctional linkers, well known to those skilled in the art, contain one end having a first reactive functionality to specifically link a first molecule and an opposite end having a second reactive functionality to specifically link to a second molecule. Depending on such factors as the molecules to be linked and the conditions in which the method of detection is performed, the linker can vary in length and composition for optimizing such properties as flexibility, stability and resistance to certain chemical and/or temperature parameters.

Nucleic acid (molecule or sequence): A deoxyribonucleotide or ribonucleotide polymer or combination thereof including without limitation, cDNA, mRNA, genomic DNA, and synthetic (such as chemically synthesized) DNA or RNA. The nucleic acid can be double stranded (ds) or single stranded (ss). Where single stranded, the nucleic acid can be the sense strand or the antisense strand. Nucleic acids can include natural nucleotides (such as A, T/U, C, and G), and can include analogs of natural nucleotides, such as labeled nucleotides. In some examples, a nucleic acid is an influenza nucleic acid, which can include nucleic acids purified from an influenza virus as well as the amplification products of such nucleic acids or synthetically produced nucleic acids.

Pharmaceutical agent: A chemical compound or composition capable of inducing a desired therapeutic or prophylactic effect when properly administered to a subject or a cell.

Pharmaceutically acceptable carriers: The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic compounds or molecules, such as one or more of the disclosed vaccines, and additional pharmaceutical agents. The term “pharmaceutically acceptable carrier” should be distinguished from “carrier” as described above in connection with an antigen/carrier conjugate.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide: A polymer in which the monomers are amino acid residues which are joined together through amide bonds. When the amino acids are alpha-amino acids, either the L-optical isomer or the D-optical isomer can be used. The term “polypeptide” is specifically intended to cover naturally occurring proteins, as well as those which are recombinantly or synthetically produced. The term “residue” or “amino acid residue” includes reference to an amino acid that is incorporated into a protein, polypeptide, or peptide.

Promoter: A promoter is an array of nucleic acid control sequences that directs transcription of a nucleic acid. A promoter includes necessary nucleic acid sequences near the start site of transcription, such as in the case of a polymerase II type promoter (a TATA element). A promoter also optionally includes distal enhancer or repressor elements which can be located as much as several thousand base pairs from the start site of transcription. Both constitutive and inducible promoters are included (see e.g., Bitter et al., Methods in Enzymology 153:516-544, 1987).

Specific, non-limiting examples of promoters include promoters derived from the genome of mammalian cells (e.g., metallothionein promoter) or from mammalian viruses (e.g., the cytomegalovirus immediate early gene promoter, the retrovirus long terminal repeat; the adenovirus late promoter; the vaccinia virus 7.5K promoter) may be used. Promoters produced by recombinant DNA or synthetic techniques may also be used. A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Protein: A molecule, particularly a polypeptide, comprised of amino acids.

Purified: The term “purified” does not require absolute purity; rather, it is intended as a relative term. Thus, for example, a purified peptide, protein, disclosed conjugate, antigen, or other active compound is one that is isolated in whole or in part from proteins or other contaminants. Generally, substantially purified peptides, proteins, conjugates, or other active compounds for use within the disclosure comprise more than 80% of all macromolecular species present in a preparation prior to admixture or formulation of the peptide, protein, conjugate or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, conjugate or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques. In one embodiment, a preparation is purified such that the specified component represents at least 50% (such as, but not limited to, 70%, 80%, 90%, 95%, 98% or 99%) of the total preparation by weight or volume.

Recombinant: A recombinant nucleic acid is one that has a sequence that is not naturally occurring or has a sequence that is made by an artificial combination of two otherwise separated segments of sequence. Recombinant also can refer to the protein (“recombinant protein”, such as recombinant diphtheria toxin) produced from a recombinant nucleic acid.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Once aligned, the number of matches is determined by counting the number of positions where an identical nucleotide or amino acid residue is present in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100.

Toxoid: A nontoxic derivative of a bacterial exotoxin produced, for example, by formaldehyde or other chemical treatment. Toxoids are useful in the formulation of immunogenic compositions because they retain most of the antigenic properties of the toxins from which they were derived.

Vaccine: A vaccine is a pharmaceutical composition that elicits a prophylactic or therapeutic immune response in a subject. In some cases, the immune response is a protective response. Typically, a vaccine elicits an antigen-specific immune response to an antigen of a pathogen, for example, a bacterial or viral pathogen. A vaccine may include a polynucleotide, a peptide or polypeptide, a polysaccharide, a virus, a bacteria, a cell or one or more cellular constituents. In some cases, the virus, bacteria or cell may be inactivated or attenuated to prevent or reduce the likelihood of disease, while maintaining the immunogenicity of the vaccine constituent. In several embodiments, a vaccine includes an immunogenic conjugate as described herein.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker gene and other genetic elements known in the art. The term vector includes plasmids, linear nucleic acid molecules.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which a disclosed invention pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999. In addition, the materials, methods, and examples are illustrative only and not intended to be limiting.

### III. Description of Several Embodiments

A. Immunogenic Conjugates

Disclosed are immunogenic conjugates that include between about 9 and about 20 amino acids (such as about 8, about 9, about 10, about 11, about 12, about 13, about 14, about 15, about 16, about 17, about 18, about 19, or about 20 amino acids) from the N-terminus of the HA2 protein from influenza (an HA2 fusion peptide), such as influenza H5N1. The HA2 fusion peptide is covalently linked to a carrier. The linkages can be at the C-terminus or the N-terminus of the HA2 fusion peptide. The conjugates have the general formula:

HA2-XXX-Pr,

where HA2 is the influenza HA2 fusion peptide, XXX is a linker and Pr is the carrier.

In some examples, the HA2 fusion peptide and the carrier protein are linked by a linker between a lysine amino acid residue present on the carrier protein and a cysteine amino acid residue present on the HA2 fusion peptide and the conjugates have the formula:

HA2-Cys-S—XXX—NH-Lys-Pr,

where HA2 is the influenza HA2 fusion peptide, XXX is a linker; Cys is a cysteine amino acid residue; S is the sulfur present in the cysteine amino acid residue; NH is the amine group present in a lysine residue; Lys is a lysine amino acid residue and Pr is the carrier protein.

In some examples, the HA2 fusion peptide and the carrier protein are linked by a thioether linkage between a lysine amino acid residue on the carrier protein and a cysteine amino acid residue present on the HA2 fusion peptide and have the formula:

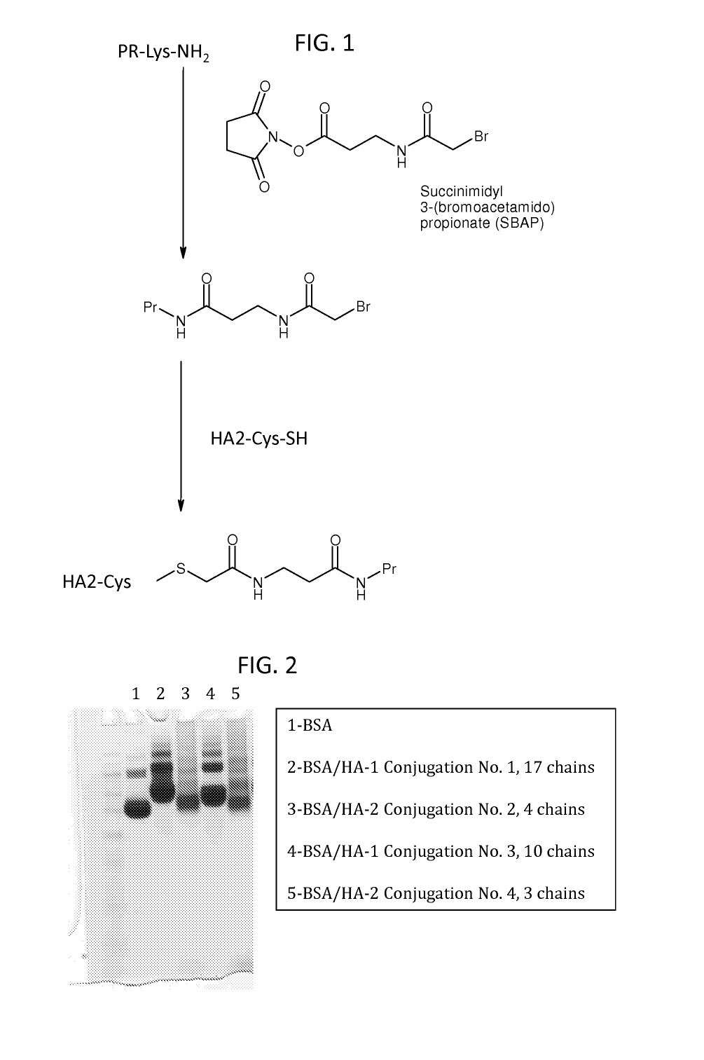

HA2-Cys-S—CH₂—C(O)—NH—CH₂—CH₂—C(O)—NH-Lys-Pr,

where HA2 is the influenza HA2 fusion peptide; Cys is a cysteine amino acid residue; S is the sulfur present in the cysteine amino acid residue; CH₂—CO—NH—CH₂—CH₂—CO is the linking group; NH is the amine group present in a lysine residue; Lys is a lysine amino acid residue and Pr is the carrier protein. A generalized scheme for introducing a thioether linkage between a cysteine amino acid residue and a lysine amino acid residue is described in Kubler-Kielb et al. Infection and Immunity 74(8): 4744-4749, 2006, which is incorporated herein by reference in its entirety. Exemplary schemes for the conjugation of an HA2 fusion peptide to a carrier protein via a thioether linkage is shown in FIG. 1

Suitable linkers are well known to those of skill in the art and include, but are not limited to, straight or branched-chain carbon linkers, heterocyclic carbon linkers or peptide linkers. One skilled in the art will recognize, for an immunogenic conjugate from two or more constituents, each of the constituents will contain the necessary reactive groups. Representative combinations of such groups are amino with carboxyl to form amide linkages or carboxy with hydroxyl to form ester linkages or amino with alkyl halides to form alkylamino linkages or thiols with thiols to form disulfides or thiols with maleimides or alkylhalides to form thioethers. Obviously, hydroxyl, carboxyl, amino and other functionalities, where not present may be introduced by known methods. Likewise, as those skilled in the art will recognize, a wide variety of linking groups may be employed. In some cases, the linking group can be designed to be either hydrophilic or hydrophobic in order to enhance the desired binding characteristics of the ligand and the receptor. The covalent linkages should be stable relative to the solution conditions under which the ligand and linking group are subjected.

Where the receptor-binding agents are polypeptides, the linkers may be joined to the constituent amino acids through their side groups (such as through a disulfide linkage to cysteine) or to the alpha carbon amino and carboxyl groups of the terminal amino acids. Alternatively, where the receptor-binding agents are polypeptides, the linker and both receptor-binding agents can be encoded as a single fusion polypeptide such that the target receptor-binding agent and the internalizing receptor-binding agent are joined by peptide bonds.

The procedure for attaching a molecule to a polypeptide varies according to the chemical structure of the molecule. Polypeptides typically contain a variety of functional groups; for example, carboxylic acid (COOH), free amine (—NH₂) or sulfhydryl (—SH) groups, which are available for reaction with a suitable functional group on a polypeptide. Alternatively, the polypeptide is derivatized to expose or attach additional reactive functional groups. The derivatization may involve attachment of any of a number of linker molecules such as those available from Pierce Chemical Company, Rockford, Ill.

As detailed in the Examples below, such an immunogenic conjugate is capable of eliciting an immune response in a subject. The HA2 fusion peptide and carrier shall be described in more detail below. Any specific combination of HA2 fusion peptide and carrier may be selected from the specific HA2 fusion peptide and carriers that are listed below.

To allow for the conjugation of the HA2 fusion peptide to carrier proteins containing a lysine amino acid residue via a thioether linkage, a cysteine residue is engineered into the HA2 fusion peptide, either at the C-terminus or the N-terminus. To allow for the conjugation of the HA2 fusion peptide to carrier proteins containing a cysteine amino acid residue via a thioether linkage, a lysine residue is engineered into the HA2 fusion peptide, either at the C-terminus or the N-terminus.

In some embodiments, a HA2 fusion peptide includes at least 9 residues (such as at least 9, 10, 11, 12, 13, 14, 15, 16, 17 or 18 residues) of the amino acid sequence set forth as GLFGAIAGFIENGWEGMI (SEQ ID NO: 1). In some embodiments, a HA2 fusion peptide includes at least residues 1-9 (such as residues 1-9, 1-10, 1-11, 1-12, 1-13, 1-14, 1-15, 1-16, 1-17 or 1-18) of the amino acid sequence set forth as GLFGAIAGFIENGWEGMI (SEQ ID NO: 1). In some embodiments, a HA2 fusion peptide includes the amino acid sequence set forth as GLFGAIAGF (residues 1-9 of SEQ ID NO: 1). In some embodiments, a HA2 fusion peptide includes the amino acid sequence set forth as GLFGAIAGFI (residues 1-10 of SEQ ID NO: 1). In some embodiments, a HA2 fusion peptide includes the amino acid sequence set forth as GLFGAIAGFIE (residues 1-11 of SEQ ID NO: 1). In some embodiments, a HA2 fusion peptide includes the amino acid sequence set forth as GLFGAIAGFIEN (residues 1-12 of SEQ ID NO: 1). In some embodiments, a HA2 fusion peptide includes the amino acid sequence set forth as GLFGAIAGFIENG (residues 1-13 of SEQ ID NO: 1). In some embodiments, a HA2 fusion peptide includes the amino acid sequence set forth as GLFGAIAGFIENGW (residues 1-14 of SEQ ID NO: 1). In some embodiments, a HA2 fusion peptide includes the amino acid sequence set forth as GLFGAIAGFIENGWE (residues 1-15 of SEQ ID NO: 1). In some embodiments, a HA2 fusion peptide includes the amino acid sequence set forth as GLFGAIAGFIENGWEG (residues 1-16 of SEQ ID NO: 1). In some embodiments, a HA2 fusion peptide includes the amino acid sequence set forth as GLFGAIAGFIENGWEGM (residues 1-17 of SEQ ID NO: 1). In some embodiments, a HA2 fusion peptide includes the amino acid sequence set forth as GLFGAIAGFIENGWEGMI (residues 1-18 of SEQ ID NO: 1).

In several embodiments, the HA2 fusion peptides included in the disclosed immunogenic conjugates are particularly hydrophobic. Thus in some embodiments, the disclosed immunogenic conjugates include one or more sequences of basic amino acids, such as a sequence of basic amino acids at the N-terminus, C-terminus or both the N- and C-terminus of the HA2 fusion peptide, for example to improve solubility. Examples of sequences of basic amino acids include amino acid sequences that are all arginine, are all lysine, or include both arginine and lysine. The disclosed sequences of basic amino acids are typically between about 2 and about 20 amino acid in length, such as about 2, about 3, about 4, about 5, about 6, about 7 about 8, about 9, about 10, about 11, about 12, about 13, about 14, about 15, about 16, about 17, about 18, about 19 and about 20 amino acids in length, for example between about 2 and about 5, about 2 and about 10, about 5 and about 15 and about 10 and about 20 amino acids in length. In some examples, a sequence of basic amino acids includes the amino acid sequence KK, KKK, KKKK (SEQ ID NO: 2), KKKKK (SEQ ID NO: 3), RR, RRR, RRRR (SEQ ID NO: 4), RRRRR (SEQ ID NO: 5), RKR, KRK, KKRK (SEQ ID NO: 6), RRKR (SEQ ID NO: 7), RKKR (SEQ ID NO: 8), KRRK (SEQ ID NO: 9), or any combination thereof. In some examples, the basic amino acid sequence is a linker between the HA2 fusion peptide and the lysine or cysteine residue that in covalently linked through a thioether linkage to the carrier. In some embodiments, such a linker includes the amino acid sequence CKK, CKKK (SEQ ID NO: 10), CKKKK (SEQ ID NO: 11), CKKKKK (SEQ ID NO: 12), CRR, CRRR (SEQ ID NO: 13), CRRRR (SEQ ID NO: 14), CRRRRR (SEQ ID NO: 15), CRKR (SEQ ID NO: 16), CKRK (SEQ ID NO: 17), CKKRK (SEQ ID NO: 18), CRRKR (SEQ ID NO: 19), CRKKR (SEQ ID NO: 20), CKRRK (SEQ ID NO: 21), KKC, KKKC (SEQ ID NO: 22), KKKKC (SEQ ID NO: 23), KKKKKC (SEQ ID NO: 24), RRC, RRRC (SEQ ID NO: 25), RRRRC (SEQ ID NO: 26), RRRRRC (SEQ ID NO: 27), RKRC (SEQ ID NO: 28), KRKC (SEQ ID NO: 29), KKRKC (SEQ ID NO: 30), RRKRC (SEQ ID NO: 31), RKKRC (SEQ ID NO: 32) or KRRKC (SEQ ID NO: 33). In some embodiments, a HA2 fusion peptide includes the amino acid sequence set forth as RKKRGLFGAIAGFIE (SEQ ID NO: 34). In specific examples, a HA2 fusion peptide includes the sequence GLFGAIAGFKKC (SEQ ID NO: 35), GLFGAIAGFIENGWEGMIKKKC (SEQ ID NO: 36) or RKKRGLFGAIAGFKKC (SEQ ID NO: 37).

It can be advantageous to produce conjugates in which more than one HA2 fusion peptide is conjugated to a single carrier protein, for example multiple copies of an HA2 fusion peptide a single amino acid sequence or several different HA2 fusion peptides with different amino acid sequences (or multiple copies of several different HA2 fusion peptides). The conjugation of multiple HA2 fusion peptides to a single carrier protein is possible because the carrier protein has multiple lysine or cysteine side-chains that can serve as sites of attachment. The amount of HA2 fusion peptide reacted with the amount of carrier may vary depending upon the specific HA2 peptide and the carrier protein. However, the respective amounts should be sufficient to introduce about 1-30 chains of HA2 fusion peptide onto the carrier protein. The resulting number of HA2 fusion peptides bound to a single protein carrier molecule may vary depending upon the specific HA2 and the carrier protein, but in general, about 1 to about 30, such as about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30 or even more than 20 HA2 fusion peptides chains can be bound to each carrier protein molecule. Thus, the average ratio of HA2 fusion peptide molecules to carrier protein molecules is between about 1:1 and about 30:1, such as about 2:1, about 3:1, about 4:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1, about 21:1, about 22:1, about 23:1, about 24:1, about 25:1, about 26:1, about 27:1, about 28:1, about 29:1, or about 30:1, for example, between about 1:1 and about 15:1, between about 5:1 and about 20:1, or between about 10:1 and about 30:1.

Examples of suitable carriers are those that can increase the immunogenicity of the conjugate and/or elicit antibodies against the carrier which are diagnostically, analytically, and/or therapeutically beneficial. Useful carriers include polymeric carriers, which can be natural, recombinantly produced, semi-synthetic or synthetic materials containing one or more amino groups, such as those present in a lysine amino acid residue present in the carrier, to which a reactant moiety can be attached. Carriers that fulfill these criteria are generally known in the art (see, for example, Fattom et al., Infect. Immun. 58:2309-12, 1990; Devi et al., PNAS 88:7175-79, 1991; Szu et al., Infect. Immun. 59:4555-61, 1991; Szu et al., J. Exp. Med. 166:1510-24, 1987; and Pavliakova et al., Infect. Immun. 68:2161-66, 2000). A carrier can be useful even if the antibody that it induces is not of benefit by itself.

Specific, non-limiting examples of suitable polypeptide carriers include, but are not limited to, natural, semi-synthetic or synthetic polypeptides or proteins from bacteria or viruses. In one embodiment, bacterial products for use as carriers include bacterial toxins. Bacterial toxins include bacterial products that mediate toxic effects, inflammatory responses, stress, shock, chronic sequelae, or mortality in a susceptible host. Specific, non-limiting examples of bacterial toxins include, but are not limited to: B. anthracis PA (for example, as encoded by bases 143779 to 146073 of GENBANK® Accession No. NC 007322), including variants that share at least 90%, at least 95%, or at least 98% amino acid sequence homology to PA, fragments that contain at least one antigenic epitope, and analogs or derivatives capable of eliciting an immune response; B. anthracis LF (for example, as encoded by the complement of bases 149357 to 151786 of GENBANK® Accession No. NC 007322); bacterial toxins and toxoids, such as tetanus toxin/toxoid (for example, as described in U.S. Pat. Nos. 5,601,826 and 6,696,065); diphtheria toxin/toxoid (for example, as described in U.S. Pat. Nos. 4,709,017 and 6,696,065), detoxified mutant diphtheria toxin, for example the genetically detoxified diphtheria toxin wherein the histidine at position 21 is replaced with glycine (DT-H21G) as described by Kossaczka et al. Infect Immun. 2000 September; 68(9): 5037-5043; P. aeruginosa exotoxin/toxoid (for example, as described in U.S. Pat. Nos. 4,428,931, 4,488,991 and 5,602,095); pertussis toxin/toxoid (for example, as described in U.S. Pat. Nos. 4,997,915, 6,399,076 and 6,696,065); and C. perfringens exotoxin/toxoid (for example, as described in U.S. Pat. Nos. 5,817,317 and 6,403,094) C. difficile toxin B or A, or analogs or mimetics of and combinations of two or more thereof. Viral proteins, such as hepatitis B surface antigen (for example, as described in U.S. Pat. Nos. 5,151,023 and 6,013,264) and core antigen (for example, as described in U.S. Pat. Nos. 4,547,367 and 4,547,368) can also be used as carriers, as well as proteins from higher organisms such as keyhole limpet hemocyanin, horseshoe crab hemocyanin, edestin, mammalian serum albumins, and mammalian immunoglobulins. In some examples, the carrier is bovine serum albumin. Exemplary methods for the conjugation of HA2 fusion peptides with carriers are described in the Examples and specifically Example 1. In a specific example, the HA2 fusion peptide is conjugated to detoxified mutant diphtheria toxin, wherein the histidine at position 21 is replaced with glycine (DT-H21G).

Following conjugation of the HA2 fusion peptide to a carrier protein, the conjugate can be purified by a variety of techniques well known to one of skill in the art. One goal of the purification step is to remove the unbound HA2 or carrier from the conjugate. One method for purification, involving ultrafiltration in the presence of ammonium sulfate, is described in U.S. Pat. No. 6,146,902. Alternatively, the conjugates can be purified away from unreacted hapten/antigen and carrier by any number of standard techniques including, for example, size exclusion chromatography, density gradient centrifugation, hydrophobic interaction chromatography, or ammonium sulfate fractionation. See, for example, Anderson et al., J. Immunol. 137:1181-86, 1986 and Jennings & Lugowski, J. Immunol. 127:1011-18, 1981. The compositions and purity of the conjugates can be determined by GLC-MS and MALDI-TOF spectrometry.

The disclosed immunogenic conjugates can be formulated into immunogenic composition (such as vaccines), for example by the addition of a pharmaceutically acceptable carrier and/or adjuvant. The formulation of immunogenic compositions is detailed below in subsection B.

B. Therapeutic Formulations.

The immunogenic compositions or vaccines disclosed herein may be included in pharmaceutical compositions (including therapeutic and prophylactic formulations), typically combined together with one or more pharmaceutically acceptable vehicles and, optionally, other therapeutic ingredients (for example, antibiotics or antiviral drugs).

Such pharmaceutical compositions can be administered to subjects by a variety of mucosal administration modes, including by oral, rectal, intranasal, intrapulmonary, or transdermal delivery, or by topical delivery to other surfaces. Optionally, the immunogenic compositions can be administered by non-mucosal routes, including by intramuscular, subcutaneous, intravenous, intra-arterial, intra-articular, intraperitoneal, or parenteral routes.

To formulate the pharmaceutical compositions, the vaccine or immunogenic conjugate can be combined with various pharmaceutically acceptable additives, as well as a base or vehicle for dispersion of the conjugate. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, TWEEN® 80), solubility enhancing agents (for example, cyclodextrins and derivatives thereof), stabilizers (for example, serum albumin), and reducing agents (for example, glutathione) can be included. Adjuvants, such as aluminum hydroxide (ALHYDROGEL®, available from Brenntag Biosector, Copenhagen, Denmark and AMPHOGEL®, Wyeth Laboratories, Madison, N.J.), Freund's adjuvant, MPL™ (3-O-deacylated monophosphoryl lipid A; Corixa, Hamilton, Ind.) and IL-12 (Genetics Institute, Cambridge, Mass.), among many other suitable adjuvants well known in the art, can be included in the compositions.

When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

The vaccine or immunogenic conjugate can be dispersed in a base or vehicle, which can include a hydrophilic compound having a capacity to disperse the vaccine, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, microspheres and films, for examples for direct application to a mucosal surface.

The vaccine or immunogenic conjugate can be combined with the base or vehicle according to a variety of methods, and release of the vaccine can be by diffusion, disintegration of the vehicle, or associated formation of water channels. In some circumstances, the vaccine or immunogenic conjugate is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43:1-5, 1991), and dispersed in a biocompatible dispersing medium, which yields sustained delivery and biological activity over a protracted time.

The compositions of the disclosure can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions for administering the vaccine or immunogenic conjugate can also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the vaccine can be brought about by including in the composition an agent which delays absorption, for example, monostearate salts and gelatin.

In certain embodiments, the vaccine or immunogenic conjugate can be administered in a time-release formulation, for example in a composition that includes a slow release polymer. These compositions can be prepared with vehicles that will protect against rapid release, for example a controlled release vehicle such as a polymer, microencapsulated delivery system or bioadhesive gel. Prolonged delivery in various compositions of the disclosure can be brought about by including in the composition agents that delay absorption, for example, aluminum monostearate hydrogels and gelatin. When controlled release formulations are desired, controlled release binders suitable for use in accordance with the disclosure include any biocompatible controlled release material which is inert to the active agent and which is capable of incorporating the vaccine and/or other biologically active agent. Numerous such materials are known in the art. Useful controlled-release binders are materials that are metabolized slowly under physiological conditions following their delivery (for example, at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

Exemplary polymeric materials for use in the present disclosure include, but are not limited to, polymeric matrices derived from copolymeric and homopolymeric polyesters having hydrolyzable ester linkages. A number of these are known in the art to be biodegradable and to lead to degradation products having no or low toxicity. Exemplary polymers include polyglycolic acids and polylactic acids, poly(DL-lactic acid-co-glycolic acid), poly(D-lactic acid-co-glycolic acid), and poly(L-lactic acid-co-glycolic acid). Other useful biodegradable or bioerodable polymers include, but are not limited to, such polymers as poly(epsilon-caprolactone), poly(epsilon-aprolactone-CO-lactic acid), poly(epsilon.-aprolactone-CO-glycolic acid), poly(beta-hydroxy butyric acid), poly(alkyl-2-cyanoacrilate), hydrogels, such as poly(hydroxyethyl methacrylate), polyamides, poly(amino acids) (for example, L-leucine, glutamic acid, L-aspartic acid and the like), poly(ester urea), poly(2-hydroxyethyl DL-aspartamide), polyacetal polymers, polyorthoesters, polycarbonate, polymaleamides, polysaccharides, and copolymers thereof. Many methods for preparing such formulations are well known to those skilled in the art (see, for example, Sustained and Controlled Release Drug Delivery Systems, J. R. Robinson, ed., Marcel Dekker, Inc., New York, 1978). Other useful formulations include controlled-release microcapsules (U.S. Pat. Nos. 4,652,441 and 4,917,893), lactic acid-glycolic acid copolymers useful in making microcapsules and other formulations (U.S. Pat. Nos. 4,677,191 and 4,728,721) and sustained-release compositions for water-soluble peptides (U.S. Pat. No. 4,675,189).

The pharmaceutical compositions of the disclosure typically are sterile and stable under conditions of manufacture, storage and use. Sterile solutions can be prepared by incorporating the conjugate in the required amount in an appropriate solvent with one or a combination of ingredients enumerated herein, as required, followed by filtered sterilization. Generally, dispersions are prepared by incorporating the vaccine and/or other biologically active agent into a sterile vehicle that contains a basic dispersion medium and the required other ingredients from those enumerated herein. In the case of sterile powders, methods of preparation include vacuum drying and freeze-drying which yields a powder of the vaccine plus any additional desired ingredient from a previously sterile-filtered solution thereof. The prevention of the action of microorganisms can be accomplished by various antibacterial and antifungal agents, for example, parabens, chlorobutanol, phenol, sorbic acid, thimerosal, and the like.

C. Methods of Treatment

In accordance with the various treatment methods of the disclosure, the disclosed immunogenic compositions or vaccines can be delivered to a subject in a manner consistent with conventional methodologies associated with management of the disorder for which treatment or prevention is sought, for example infection from influenza virus. In accordance with the disclosure herein, a prophylactically or therapeutically effective amount of the immunogenic composition and/or other biologically active agent is administered to a subject in need of such treatment for a time and under conditions sufficient to prevent, inhibit, and/or ameliorate a selected disease or condition or one or more symptom(s) thereof. In some embodiments, administration of the disclosed vaccines to a subject elicits an immune response against an influenza antigenic epitope in the subject, for example an immune response against an influenza HA protein. In some embodiments, a subject is selected for treatment that has, or is at risk for developing, an influenza infection, for example because of exposure or the possibility of exposure to influenza. Alternatively, the subject is selected because of risk factors for infection and/or morbidity (for example the subject is very young or old, pregnant, immunocompromised or suffering from a chronic pulmonary condition)

Typical subjects intended for treatment with the compositions and methods of the present disclosure include humans, as well as non-human primates and other animals To identify subjects for prophylaxis or treatment according to the methods of the disclosure, accepted screening methods are employed to determine risk factors associated with a targeted or suspected disease of condition (for example, coughing disease), or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods, which are available and well known in the art to detect and/or characterize influenza infection. These and other routine methods allow the clinician to select patients in need of therapy using the methods and pharmaceutical compositions of the disclosure. In accordance with these methods and principles, a vaccine and/or other biologically active agent can be administered according to the teachings herein as an independent prophylaxis or treatment program, or as a follow-up, adjunct or coordinate treatment regimen to other treatments.

The vaccine or immunogenic conjugate can be used in coordinate vaccination protocols or combinatorial formulations. In certain embodiments, novel combinatorial immunogenic compositions and coordinate immunization protocols employ separate immunogens or formulations, each directed toward eliciting an anti-influenza immune response, such as an immune response to influenza HA protein. Separate vaccines that elicit the anti-influenza immune response can be combined in a polyvalent immunogenic composition administered to a subject in a single immunization step, or they can be administered separately (in monovalent immunogenic compositions) in a coordinate immunization protocol.

The administration of the vaccines and/or immunogenic conjugates of the disclosure can be for either prophylactic or therapeutic purpose. When provided prophylactically, the vaccine and/or immunogenic conjugate is provided in advance of any symptom, for example in advance of infection, such as in the form of a yearly flu shot. The prophylactic administration of the vaccine and/or immunogens serves to prevent or ameliorate any subsequent infection. When provided therapeutically, the vaccine and/or immunogenic conjugate is provided at (or shortly after) the onset of a symptom of disease or infection. The vaccine and/or immunogenic conjugate of the disclosure can thus be provided prior to the anticipated exposure to influenza virus so as to attenuate the anticipated severity, duration or extent of an infection and/or associated disease symptoms, after exposure or suspected exposure to the virus, or after the actual initiation of an infection.

For prophylactic and therapeutic purposes, the vaccine and/or immunogenic conjugate can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the vaccine and/or immunogenic conjugate can be provided as repeated doses within a prolonged prophylaxis or treatment regimen that will yield clinically significant results to alleviate one or more symptoms or detectable conditions associated with a targeted disease or condition as set forth herein. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, porcine, feline, ferret, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer a therapeutically effective amount of the vaccine and/or immunogenic conjugate (for example, amounts that are effective to elicit a desired immune response or alleviate one or more symptoms of a targeted disease). In alternative embodiments, an effective amount or effective dose of the vaccine and/or immunogenic conjugate may simply inhibit or enhance one or more selected biological activities correlated with a disease or condition, as set forth herein, for either therapeutic or diagnostic purposes.

The actual dosage of the vaccine and/or immunogenic conjugate will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the vaccine for eliciting the desired activity or biological response in the subject. Dosage regimens can be adjusted to provide an optimum prophylactic or therapeutic response. As described above in the forgoing listing of terms, an effective amount is also one in which any toxic or detrimental side effects of the vaccine and/or other biologically active agent is outweighed in clinical terms by therapeutically beneficial effects. A non-limiting range for a therapeutically effective amount of a vaccine and/or other biologically active agent within the methods and formulations of the disclosure is about 0.01 mg/kg body weight to about 10 mg/kg body weight, such as about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.5 mg/kg, about 2 mg/kg, about 2.5 mg/kg, about 3 mg/kg, about 4 mg/kg, about 5 mg/kg, or about 10 mg/kg, for example 0.01 mg/kg to about 1 mg/kg body weight, about 0.05 mg/kg to about 5 mg/kg body weight, about 0.2 mg/kg to about 2 mg/kg body weight, or about 1.0 mg/kg to about 10 mg/kg body weight.

Upon administration of a vaccine and/or immunogenic conjugate of this disclosure (for example, via injection, aerosol, oral, topical or other route), the immune system of the subject typically responds to the immunogenic composition by producing antibodies specific for influenza proteins, such as to the HA protein and/or an antigenic epitope presented by the vaccine and/or immunogenic conjugate. Such a response signifies that an immunologically effective dose of the vaccine was delivered. An immunologically effective dosage can be achieved by single or multiple administrations (including, for example, multiple administrations per day), daily, or weekly administrations. For each particular subject, specific dosage regimens can be evaluated and adjusted over time according to the individual need and professional judgment of the person administering or supervising the administration of the vaccine. In some embodiments, the antibody response of a subject administered the compositions of the disclosure will be determined in the context of evaluating effective dosages/immunization protocols. In most instances it will be sufficient to assess the antibody titer in serum or plasma obtained from the subject. Decisions as to whether to administer booster inoculations and/or to change the amount of the composition administered to the individual can be at least partially based on the antibody titer level. The antibody titer level can be based on, for example, an immunobinding assay which measures the concentration of antibodies in the serum which bind to a specific antigen, for example, influenza HA protein.

Dosage can be varied by the attending clinician to maintain a desired concentration at a target site (for example, the lungs or systemic circulation). Higher or lower concentrations can be selected based on the mode of delivery, for example, trans-epidermal, rectal, oral, pulmonary, or intranasal delivery versus intravenous or subcutaneous delivery. Dosage can also be adjusted based on the release rate of the administered formulation, for example, of an intrapulmonary spray versus powder, sustained release oral versus injected particulate or transdermal delivery formulations, and so forth. To achieve the same serum concentration level, for example, slow-release particles with a release rate of 5 nanomolar (under standard conditions) would be administered at about twice the dosage of particles with a release rate of 10 nanomolar.

The methods of using vaccines and/or immunogenic conjugates, and the related compositions and methods of the disclosure are useful in increasing resistance to, preventing, ameliorating, and/or treating infection and disease caused by influenza virus in animal hosts, and other, in vitro applications. These immunogenic compositions can be used for active immunization for prevention of infection, and for preparation of immune antibodies. The immunogenic compositions are composed of non-toxic components, suitable for infants, children of all ages, and adults.

This disclosure also includes kits, packages and multi-container units containing the herein described pharmaceutical compositions, active ingredients, and/or means for administering the same for use in the prevention and treatment of influenza and other conditions in mammalian subjects. Kits for diagnostic use are also provided. In one embodiment, these kits include a container or formulation that contains one or more of the conjugates described herein. In one example, this component is formulated in a pharmaceutical preparation for delivery to a subject. The vaccine is optionally contained in a bulk dispensing container or unit or multi-unit dosage form. Optional dispensing means can be provided, for example a pulmonary or intranasal spray applicator. Packaging materials optionally include a label or instruction indicating for what treatment purposes and/or in what manner the pharmaceutical agent packaged therewith can be used.

D. Peptide or Protein Production

Nucleic acid molecules encoding the HA2 fusion peptides HA2 fusion peptides and carrier proteins can be prepared by chemical peptide synthesis. Nucleic acid molecules encoding the HA2 fusion peptides HA2 fusion peptides and carrier proteins, and any other peptides or proteins of this disclosure can be prepared by cloning techniques. Examples of appropriate cloning and sequencing techniques, and instructions sufficient to direct persons of skill through many cloning exercises are found in Sambrook et al., Molecular Cloning: A Laboratory Manual (2nd Ed.), Vols. 1-3, Cold Spring Harbor Laboratory (1989), Berger and Kimmel (eds.), Guide to Molecular Cloning Techniques, Academic Press, Inc., San Diego Calif. (1987), or Ausubel et al. (eds.), Current Protocols in Molecular Biology, Greene Publishing and Wiley-Interscience, NY (1987). Product information from manufacturers of biological reagents and experimental equipment also provide useful information. Such manufacturers include the SIGMA chemical company (Saint Louis, Mo.), R&D systems (Minneapolis, Minn.), Pharmacia LKB Biotechnology (Piscataway, N.J.), CLONTECH® laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), INVITROGEN™ (San Diego, Calif.), and Applied Biosystems (Foster City, Calif.), as well as many other commercial sources known to one of skill.

In some embodiments, the peptides of this disclosure are produced recombinantly, for example from cells transformed or transfected with polynucleotides encoding the peptides or portion thereof. Methods for the manipulation and insertion of the nucleic acids encoding the peptides of this disclosure or portions thereof into vectors for the expression of polypeptides are well known in the art (see for example, Sambrook et al., Molecular Cloning, a Laboratory Manual, 2d edition, Cold Spring Harbor Press, Cold Spring Harbor, N.Y., 1989, and Ausubel et al., Current Protocols in Molecular Biology, Greene Publishing Associates and John Wiley & Sons, New York, N.Y., 1994).

The nucleic acid sequences encoding immunogenic fragments of HA peptides, carrier proteins, and any other peptides or proteins of this disclosure can be operatively linked to expression control sequences. An expression control sequence operatively linked to a coding sequence is ligated such that expression of the coding sequence is achieved under conditions compatible with the expression control sequences. The expression control sequences include, but are not limited to appropriate promoters, enhancers, transcription terminators, a start codon (for instance, ATG) in front of a protein-encoding gene, splicing signal for introns, maintenance of the correct reading frame of that gene to permit proper translation of mRNA, and stop codons.

Transformation of a host cell with recombinant DNA may be carried out by conventional techniques as are well known to those skilled in the art. Where the host is prokaryotic, such as E. coli, competent cells, which are capable of DNA uptake can be prepared from cells harvested after exponential growth phase and subsequently treated by the CaCl₂ method using procedures well known in the art. Alternatively, MgCl₂, or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors may be used. Eukaryotic cells can also be cotransformed with polynucleotide sequences encoding HA2 fusion peptides and/or carrier proteins and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

The expression and purification of any of these immunogenic fragments of HA peptides, carrier proteins, and any other peptides or proteins of this disclosure by standard laboratory techniques, is now enabled. Fragments amplified as described herein can be cloned into standard cloning vectors and expressed in commonly used expression systems consisting of a cloning vector and a cell system in which the vector is replicated and expressed. Purified proteins may be used for functional analyses, antibody production, diagnosis, and subject therapy. Partial or full-length cDNA sequences, which encode for the protein, may be ligated into bacterial expression vectors. Methods for expressing large amounts of protein from a cloned gene introduced into E. coli may be utilized for the purification of proteins.

Immunogenic fragments of HA2 fusion peptides, carrier proteins, and any other peptides or proteins of this disclosure may also be produced in E. coli in large amounts for vaccine development and/or evaluation. Standard prokaryotic cloning vectors may also be used, for example, pBR322, pUC18, or pUC19 as described in Sambrook et al. (Molecular Cloning: A Laboratory Manual, 2^{nd} ed., vol. 1-3, Cold Spring Harbor, N.Y. 1989). Nucleic acids encoding immunogenic fragments of HA2 fusion peptides, carrier proteins, and any other peptides or proteins of this disclosure may be cloned into such vectors, which may then be transformed into bacteria such as E. coli, which may then be cultured to express the protein of interest. Other prokaryotic expression systems include, for instance, the arabinose-induced pBAD expression system that allows tightly controlled regulation of expression, the IPTG-induced pRSET system that facilitates rapid purification of recombinant proteins and the IPTG-induced pSE402 system that has been constructed for optimal translation of eukaryotic genes. These three systems are available commercially from INVITROGEN™ and, when used according to the manufacturer's instructions, allow routine expression and purification of proteins.

Methods and plasmid vectors for producing proteins and peptides in bacteria are described in Sambrook et al. (Molecular Cloning: A Laboratory Manual, Cold Spring Harbor, N.Y., 1989, Chapter 17). Such proteins and peptides may be made in large amounts, are easy to purify, and can be used to elicit antibody response. Proteins and proteins can be produced in bacteria by placing a strong, regulated promoter and an efficient ribosome binding site upstream of the cloned gene. If low levels of protein are produced, additional steps may be taken to increase protein production; if high levels of protein are produced, purification is relatively easy. Suitable methods are presented in Sambrook et al. (Molecular Cloning: A Laboratory Manual, Cold Spring Harbor, N.Y., 1989) and are well known in the art. Often, proteins expressed at high levels are found in insoluble inclusion bodies. Methods for extracting proteins from these aggregates are described by Sambrook et al. (Molecular Cloning: A Laboratory Manual, Cold Spring Harbor, N.Y., 1989, Chapter 17).

Vector systems suitable for the expression of proteins and peptides include the pUR series of vectors (Ruther and Muller-Hill, EMBO J. 2:1791, 1983), pEX1-3 (Stanley and Luzio, EMBO J. 3:1429, 1984) and pMR100 (Gray et al., Proc. Natl. Acad. Sci. USA 79:6598, 1982). Vectors suitable for the production of intact native proteins include pKC30 (Shimatake and Rosenberg, Nature 292:128, 1981), pKK177-3 (Amann and Brosius, Gene 40:183, 1985) and pET-3 (Studiar and Moffatt, J. Mol. Biol. 189:113, 1986). The DNA sequence can also be transferred to other cloning vehicles, such as other plasmids, bacteriophages, cosmids, animal viruses, and yeast artificial chromosomes (YACs) (Burke et al., Science 236:806-12, 1987). These vectors may then be introduced into a variety of hosts including somatic cells, and simple or complex organisms, such as bacteria, fungi (Timberlake and Marshall, Science 244:1313-7, 1989), invertebrates, plants (Gasser and Fraley, Science 244:1293, 1989), and mammals (Pursel et al., Science 244:1281-8, 1989).

Various yeast strains and yeast-derived vectors are commonly used for expressing and purifying proteins, for example, Pichia pastoris expression systems are available from INVITROGEN™ (Carlsbad, Calif.). Such systems include suitable Pichia pastoris strains, vectors, reagents, transformants, sequencing primers and media.

Non-yeast eukaryotic vectors can also be used for expression of the HA2 fusion peptides. Examples of such systems are the well known Baculovirus system, the Ecdysone-inducible mammalian expression system that uses regulatory elements from Drosophila melanogaster to allow control of gene expression, and the Sindbis viral expression system that allows high level expression in a variety of mammalian cell lines. These expression systems are available from INVITROGEN™.

For expression in mammalian cells, the cDNA sequence may be ligated to heterologous promoters, such as the simian virus SV40, promoter in the pSV2 vector (Mulligan and Berg, 1981, Proc. Natl. Acad. Sci. USA 78:2072-6), and introduced into cells, such as monkey COS-1 cells (Gluzman, Cell 23:175-82, 1981), to achieve transient or long-term expression. The stable integration of the chimeric gene construct may be maintained in mammalian cells by biochemical selection, such as neomycin (Southern and Berg, J. Mol. Appl. Genet. 1:327-41, 1982) and mycophoenolic acid (Mulligan and Berg, Proc. Natl. Acad. Sci. USA 78:2072-6, 1981).

DNA sequences can be manipulated with standard procedures such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate or with the use of specific oligonucleotides in combination with PCR.

The cDNA sequence (or portions derived from it) or a mini gene (a cDNA with an intron and its own promoter) may be introduced into eukaryotic expression vectors by conventional techniques. These vectors are designed to permit the transcription of the cDNA eukaryotic cells by providing regulatory sequences that initiate and enhance the transcription of the cDNA and ensure its proper splicing and polyadenylation. Vectors containing the promoter and enhancer regions of the SV40 or long terminal repeat (LTR) of the Rous Sarcoma virus and polyadenylation and splicing signal from SV40 are readily available (Mulligan and Berg, Proc. Natl. Acad. Sci. USA 78:2072-6, 1981; Gorman et al., Proc. Natl. Acad. Sci USA 78:6777-81, 1982). The level of expression of the cDNA can be manipulated with this type of vector, either by using promoters that have different activities (for example, the baculovirus pAC373 can express cDNAs at high levels in S. frugiperda cells (Summers and Smith, 1985, Genetically Altered Viruses and the Environment, Fields et al. (Eds.) 22:319-328, Cold Spring Harbor Laboratory Press, Cold Spring Harbor, N.Y.) or by using vectors that contain promoters amenable to modulation, for example, the glucocorticoid-responsive promoter from the mouse mammary tumor virus (Lee et al., Nature 294:228, 1982). The expression of the cDNA can be monitored in the recipient cells 24 to 72 hours after introduction (transient expression).

In addition, some vectors contain selectable markers such as the gpt (Mulligan and Berg, Proc. Natl. Acad. Sci. USA 78:2072-6, 1981) or neo (Southern and Berg, J. Mol. Appl. Genet. 1:327-41, 1982) bacterial genes. These selectable markers permit selection of transfected cells that exhibit stable, long-term expression of the vectors (and therefore the cDNA). The vectors can be maintained in the cells as episomal, freely replicating entities by using regulatory elements of viruses such as papilloma (Sarver et al., Mol. Cell Biol. 1:486, 1981) or Epstein-Barr (Sugden et al., Mol. Cell Biol. 5:410, 1985). Alternatively, one can also produce cell lines that have integrated the vector into genomic DNA. Both of these types of cell lines produce the gene product on a continuous basis. One can also produce cell lines that have amplified the number of copies of the vector (and therefore of the cDNA as well) to create cell lines that can produce high levels of the gene product (Alt et al., J. Biol. Chem. 253:1357, 1978).

The transfer of DNA into eukaryotic, in particular human, or other mammalian cells, is now a conventional technique. The vectors are introduced into the recipient cells as pure DNA (transfection) by, for example, precipitation with calcium phosphate (Graham and vander Eb, 1973, Virology 52:466) or strontium phosphate (Brash et al., Mol. Cell Biol. 7:2013, 1987), electroporation (Neumann et al., EMBO J. 1:841, 1982), lipofection (Felgner et al., Proc. Natl. Acad. Sci USA 84:7413, 1987), DEAE dextran (McCuthan et al., J. Natl. Cancer Inst. 41:351, 1968), microinjection (Mueller et al., Cell 15:579, 1978), protoplast fusion (Schafner, Proc. Natl. Acad. Sci. USA 77:2163-7, 1980), or pellet guns (Klein et al, Nature 327:70, 1987). Alternatively, the cDNA can be introduced by infection with virus vectors. Systems are developed that use, for example, retroviruses (Bernstein et al., Gen. Engrg. 7:235, 1985), adenoviruses (Ahmad et al., J. Virol. 57:267, 1986), or Herpes virus (Spaete et al., Cell 30:295, 1982).

Using the above techniques, the expression vectors containing STLV-3 subtype D genes or cDNA sequence or fragments or variants or mutants thereof can be introduced into human cells, primate cells, mammalian cells from other species, or non-mammalian cells as desired. The choice of cell is determined by the purpose of the treatment. For example, monkey COS cells (Gluzman, Cell 23:175-82, 1981) that produce high levels of the SV40 T antigen and permit the replication of vectors containing the SV40 origin of replication may be used. Similarly, Chinese hamster ovary (CHO), mouse NIH 3T3 fibroblasts or human fibroblasts or lymphoblasts may be used.

HA2 fusion peptides and carrier proteins also may be produced, for example by chemical synthesis by any of a number of manual or automated methods of synthesis known in the art. For example, solid phase peptide synthesis (SPPS) is carried out on a 0.25 millimole (mmole) scale using an Applied Biosystems Model 431A Peptide Synthesizer and using 9-fluorenylmethyloxycarbonyl (Fmoc) amino-terminus protection, coupling with dicyclohexylcarbodiimide/hydroxybenzotriazole or 2-(1H-benzo-triazol-1-yl)-1,1,3,3-tetramethyluronium hexafluorophosphate/hydroxybenzotriazole (HBTU/HOBT), and using p-hydroxymethylphenoxymethylpolystyrene (HMP) or Sasrin resin for carboxyl-terminus acids or Rink amide resin for carboxyl-terminus amides.

Fmoc-derivatized amino acids are prepared from the appropriate precursor amino acids by tritylation and triphenylmethanol in trifluoroacetic acid, followed by Fmoc derivitization as described by Atherton et al. Solid Phase Peptide Synthesis, IRL Press: Oxford, 1989.

Sasrin resin-bound peptides are cleaved using a solution of 1% TFA in dichloromethane to yield the protected peptide. Where appropriate, protected peptide precursors are cyclized between the amino- and carboxyl-termini by reaction of the amino-terminal free amine and carboxyl-terminal free acid using diphenylphosphorylazide in nascent peptides wherein the amino acid sidechains are protected.

HMP or Rink amide resin-bound products are routinely cleaved and protected sidechain-containing cyclized peptides deprotected using a solution comprised of trifluoroacetic acid (TFA), optionally also comprising water, thioanisole, and ethanedithiol, in ratios of 100:5:5:2.5, for 0.5-3 hours at room temperature.

Crude peptides are purified by preparative high pressure liquid chromatography (HPLC), for example using a Waters Delta-Pak C18 column and gradient elution with 0.1% TFA in water modified with acetonitrile. After column elution, acetonitrile is evaporated from the eluted fractions, which are then lyophilized. The identity of each product so produced and purified may be confirmed by fast atom bombardment mass spectroscopy (FABMS) or electrospray mass spectroscopy (ESMS).

The subject matter of the present disclosure is further illustrated by the following non-limiting Examples.

### EXAMPLES

### Example 1

### Derivatization of BSA with NHJK-1 and NHJK-2 (HA Peptides)

Bovine Serum Albumin (BSA) was derivatized with the addition of N-succinimidyl 3-(bromoacetamido) propionate (SBAP, Pierce, Rockford, Ill.). 10 mg of SBAP (33 μmol) in 95 μl DMSO mg was added to 65 mg of BSA (59 μmol Lys) in 1.3 ml Buffer B, pH was 7.4. After 2 hours, reaction was passed in two runs through a G-50 column, 1×50 cm in 0.2 M NaCl. The resulting derivatized BSA was concentrated to 2.8 ml (14.5 mg/ml) a subjected to taken for Maldi TOF analysis (BSA=66.4 kDa; BSA-Br=70.8 kDa; 4300/192=22 linkers)

For the conjugation #1: GLFGAIAGFKKC (NHJK-1; SEQ ID NO: 35) (Mm 1211 Da) 7 mg of BSA-Br in Buffer B was mixed with 7 mg NHJK-1 in 0.2 ml water, pH 7.4. The resulting conjugate was purified on a G-50 column as above and concentrated to 2 milliliters. This yielded 3.2 mg/ml protein in a volume of 2 ml for a total 6.4 mg protein and 0.71 mg/ml peptide conjugated. Maldi TOF analysis indicated that 89.8 kDa-70.8 kDa=1900 Da/1131 Da=17 peptide chains per BSA molecule.

For conjugation #2: GLFGAIAGFIENGWEGMIKKKC (NHJK-2; SEQ ID NO: 36) (Mm 2370 Da, the peptide was dissolved in DMSO 7 mg of BSA-Br in Buffer B was mixed with 7 mg NHJK-2 in 0.2 ml DMSO. The resultant conjugate was purified on a G-50 column as above and concentrated to 1.8 ml. This yielded 0.53 mg/ml protein for a total 0.95 mg with 0.060 mg/ml peptide for a total of 0.109 mg. Maldi analysis indicted 79.4 kDa-70.8 kDa=8600 Da/2290 Da=4 chains per BSA molecule.

For conjugation #3: GLFGAIAGFKKC (NHJK-1; SEQ ID NO: 35) (Mm 1211 Da) 7 mg of BSA-Br in Buffer B was mixed with 4 mg NHJK-1 in 0.2 ml water, pH 7.4. The resultant was purified on a G-50 column as above and concentrated to 4 ml. This yielded 1.75 mg/ml protein in a volume of 4 ml for a total 6.8 mg protein and 0.26 mg/ml peptide conjugated. Maldi TOF analysis indicated that 82.6 kDa-70.9 kDa=1170 Da/1131 Da=10 peptide chains per BSA molecule.

For conjugation #4: GLFGAIAGFIENGWEGMIKKKC (NHJK-2; SEQ ID NO: 36) (Mm 2370, the peptide was dissolved peptide in DMSO 7 mg of BSA-Br in Buffer B was mixed with 4 mg NHJK-2 in 0.16 ml DMSO. The resultant conjugate was purified on a G-50 column as above and concentrated to 2.8 ml. This yielded 0.63 mg/ml protein for a total 1.75 mg with 0.057 mg/ml peptide. Maldi TOF analysis indicted 77.4 kDa-70.8 kDa=6600 Da/2290 Da=3 chains per BSA molecule.

The results of polyacrylamide gel electrophoresis of the 4 conjugation reactions are shown in FIG. 2. In a similar manner conjugates of HSA and NHJK-1 were prepared resulting in two products with 21 and 17 chains. These conjugates are being absorbed onto alum and non-absorb and absorbed preparations will be injected into mice.

PEPTIDE 1 as inhibitor; HA coating (NHJK-1:
GLFGAIAGFKKC, (SEQ ID NO: 35))
Serum 2300G (anti-Pep 1):

| Inh [ug/well] | OD | % Inh |
|---|---|---|
| 100 | 0.341 | 82% |
| 25 | 0.236 | 88% |
| 5 | 0.139 | 93% |
| 0 | 1.925 | — |

Serum 2300H (anti-Pep1)

| Inh [ug/well] | OD | % Inh |
|---|---|---|
| 100 | 0.324 | 82% |
| 25 | 0.273 | 85% |
| 5 | 0.171 | 91% |
| 0 | 1.845 | — |

Serum 2301F (anti-Pep2)

| Inh [ug/well] | OD | % Inh |
|---|---|---|
| 100 | 1.088 | 32% |
| 25 | 1.580 | 2% |
| 5 | 1.567 | 3% |
| 0 | 1.610 | — |

PEPTIDE 2 as inhibitor; HA coating (NHJK-2:
GLFGAIAGFIENGWEGMIKKKC, (SEQ ID NO: 36)
Serum 2300G (anti-Pep 1):

| Inh [ug/well] | OD | % inh |
|---|---|---|
| 100 | 0.261 | 86% |
| 25 | 0.182 | 90% |
| 5 | 0.121 | 93% |
| 0 | 1.881 | — |

Serum 2300H (anti-Pep 1):

| Inh [ug/well] | OD | % inh |
|---|---|---|
| 100 | 0.249 | 86% |
| 25 | 0.186 | 90% |
| 5 | 0.194 | 89% |
| 0 | 1.835 | — |

Serum 2301F (anti-Pep2)

| Inh [ug/well] | OD | % inh |
|---|---|---|
| 100 | 0.469 | 71% |
| 25 | 0.943 | 43% |
| 5 | 1.209 | 26% |
| 0 | 1.640 | — |

### Example 2

### Immunization of Mice

This example describes exemplary in vitro tests of the HA2 based vaccines.

Groups of NIH Swiss mice are injected subcutaneously with HA2 conjugate two or three times two weeks apart. Controls are injected with PBS. Blood samples are collected one week after the last immunization.

Anti-HA IgG is measured by enzyme-linked immunosorbent assay (ELISA). Nunc 96-well Flat-bottom IMMUNE® plates (Nalgene Nunc International, Rochester, N.Y.) are coated with rHA. Antigen-coated plates are washed with “ELISA washing buffer” and test and control sera added. Following overnight incubation at room temperature, the plates are washed six times with ELISA washing buffer and anti-HA specific IgG detected using goat anti-mouse IgG conjugated to alkaline phosphatase (KPL, Gaithersburg, Md.) diluted 1:1000. The plates are incubated at room temperature 4 hours and washed six times with ELISA washing buffer. One hundred microliters of freshly prepared substrate is added to each well, the enzyme-substrate reaction is carried out at room temperature for 20 minutes, and absorbance measured at 405 nm. Antibody concentration is calculated from a standard curve of mice serum raised against rHA, diluted 1:30,000 and assigned a value of 100 ELISA units (EU). The results were analyzed using an ELISA data-processing program.

Hemagglutination inhibition (HI) is performed by standard procedures in 96-well V-bottom plates using 1% (vol/vol) horse red blood cells (HRBC). Sera were pre-treated with receptor destroying enzyme, RDE (II) “Seiken” (Denka Seiken Co., Ltd., Tokyo, Japan) from Vibrio cholerae (1 volume of sera: 3 volume of RDE). After a 15 minute incubation at room temperature an equal volume of 1% (v/v) HRBC in PBS is added and the plates read after 1 hour.

TABLE 1
Levels of anti-HA (H5N1) antibodies (GMs) induced
by BSA conjugates of Peptide HA-1 (NHJK-1):
GLFGAIAGFKKC (SEQ ID NO: 35) and Peptide HA-2
(NHJK-2): GLFGAIAGFIENGWEGMIKKKC (SEQ ID NO: 36)
in mice.

| | | No of | | IgG anti- |
|---|---|---|---|---|
| Lot | PREPARATION | chains | Group | HA [EU] |
| #1 | BSA/HA-1 | 17 | 2300 | 1.1 |
| #2 | BSA/HA-2 | 4 | 2301 | 0.1 |
| #3 | BSA/HA-1 | 10 | 2302 | 0.02 |
| #10 | HSA/HA-1 | 21 | tbd | tbd |
| #10 | HSA/HA-1 on alum | 21 | tbd | tbd |
| #1 | HSA/HA-1 | 17 | tbd | tbd |
| #11 | HSA/HA-1 on alum | 17 | tbd | tbd |

IgG level based on 100 EU given to the standard; standard diluted 1: 30,000.

### Example 3

### Preparation of rDT/HA2 Conjugate Vaccine

This example describes exemplary methods for preparing a conjugate vaccine useful in the treatment and/or inhibition of influenza infection.

Ten mg of N-succinimidyl 3-(bromoacetamido) propionate (SBAP, Pierce, Rockford, Ill.) in 40 ul DMSO are added and reacted at pH 7.2 at room temp to 28 mg of recombinant diphtheria toxin (rDT) in 2 ml buffer A (0.1 M phosphate, 1 mM EDTA, 1% glycerol, pH 7.2). The resulting solution is mixed. Next, the solution is applied to a Sephadex G-50 column (1×50 cm) in phosphate buffered saline (PBS), and the void volume fraction (rDT-Br) is concentrated using an Amicon Ultra-15 centrifuge filter device (MILLIPORE®, Billerica, Mass.). To 22 mg rDT-Br HA2 is added and reacted at pH 7.2 overnight. The solution is then passed through a SEPHADEX G-75 (1×100 cm) column in PBS and the void volume fraction collected and analyzed for protein contents and molecular mass by Matrix Assisted Laser Desorption/Ionization—Time Of Flight (MALDI-TOF) mass spectrometry and sodium dodecyl sulfate polyacrylamide gel electrophoresis (SDS-PAGE).

### Example 4

### Treatment of Subjects with the Disclosed Vaccines

This example describes methods that can be used to treat a subject that has or is at risk of having an infection from influenza by administration of one or more of the disclosed vaccines. In particular examples, the method includes screening a subject having, thought to have, or at risk of having (for example due to impaired immunity, physiological status, or exposure to influenza) an influenza infection. Subjects of an unknown infection status can be examined to determine if they have an infection, for example using serological tests, physical examination, enzyme-linked immunosorbent assay (ELISA), radiological screening or other diagnostic technique known to those of ordinary skill in the art. In some examples, a subject is selected that has an influenza infection or is at risk of acquiring an influenza infection. Subjects found to (or known to) have an influenza infection and thereby treatable by administration of the disclosed vaccines are selected to receive the vaccine. Subjects may also be selected who are at risk of developing an influenza infection for example, the elderly, the immunocompromised and the very young, such as infants.

Subjects selected for treatment can be administered a therapeutic amount of disclosed vaccine. The vaccine can be administered at doses of 1 μg/kg body weight to about 1 mg/kg body weight per dose, such as 1 μg/kg body weight-100 μg/kg body weight per dose, 100 μg/kg body weight-500 μg/kg body weight per dose, or 500 μg/kg body weight-1000 μg/kg body weight per dose or even greater. However, the particular dose can be determined by a skilled clinician. The agent can be administered in several doses, for example continuously, daily, weekly, or monthly.

The mode of administration can be any used in the art. The amount of agent administered to the subject can be determined by a clinician, and may depend on the particular subject treated. Specific exemplary amounts are provided herein (but the disclosure is not limited to such doses).

While this disclosure has been described with an emphasis upon particular embodiments, it will be obvious to those of ordinary skill in the art that variations of the particular embodiments may be used, and it is intended that the disclosure may be practiced otherwise than as specifically described herein. Features, characteristics, compounds, chemical moieties, or examples described in conjunction with a particular aspect, embodiment, or example of the invention are to be understood to be applicable to any other aspect, embodiment, or example of the invention. Accordingly, this disclosure includes all modifications encompassed within the spirit and scope of the disclosure as defined by the following claims.

1. An immunogenic conjugate comprising an influenza virus hemagglutinin 2 (HA2) fusion peptide between 9 and 20 amino acids in length covalently linked to a carrier, wherein the conjugate elicits an immune response in a subject.

2. The immunogenic conjugate of claim 1, wherein the HA2 fusion peptide comprises residues 1-9 of the amino acid sequence set forth as GLFGAIAGFIENGWEGMI (SEQ ID NO: 1).

3. The immunogenic conjugate of claim 2, wherein the HA2 fusion peptide comprises the amino acid sequence set forth GLFGAIAGFIENGWEGMI (SEQ ID NO: 1).

4. The immunogenic conjugate of claim 1, wherein the carrier comprises bovine serum albumin, recombinant B. anthracis protective antigen, recombinant P. aeruginosa exotoxin A, tetanus toxoid, recombinant diphtheria toxoid, pertussis toxoid, recombinant Clostridium difficile toxin B subunit (rBRU), C. perfringens toxoid, or analogs or mimetics of and combinations of two or more thereof.

5. The immunogenic conjugate of claim 4, wherein the carrier comprises recombinant diphtheria toxoid (rDT).

6. The immunogenic conjugate of claim 5, wherein the recombinant diphtheria toxoid comprises genetically detoxified diphtheria toxin wherein the histidine at position 21 is replaced with glycine (DT-H21G).

7. The immunogenic conjugate of claim 1, wherein the HA2 fusion peptide and the carrier are covalently linked by a linker.

8. The immunogenic conjugate of claim 1, wherein the HA2 fusion peptide and the carrier are covalently linked via a thioether linkage between a lysine amino acid residue present on the carrier and a cysteine amino acid residue present on the HA2 fusion peptide.

9. The immunogenic conjugate of claim 8, wherein the lysine residue is present on the carrier and the cysteine residue is introduced at the C-terminal end or N-terminal end of the HA2 fusion peptide.

10. The immunogenic conjugate of claim 1, wherein the HA2 fusion peptide further comprises a polybasic amino acid sequence, between 2 and 20 amino acids in length.

11. The immunogenic conjugate of claim 1, wherein the HA2 fusion peptide comprises or consists of RKKRGLFGAIAGFIE (SEQ ID NO: 34), GLFGAIAGFKKC (SEQ ID NO: 35), GLFGAIAGFIENGWEGMIKKKC (SEQ ID NO: 36) or RKKRGLFGAIAGFKKC (SEQ ID NO: 37).

12. (canceled)

13. The immunogenic conjugate of claim 1, wherein the average ratio of HA2 fusion peptide molecules to carrier protein molecules is between about 1:1 and 30:1.

14. An immunogenic composition comprising the conjugate of claim 1 and a pharmaceutically acceptable carrier.

15. The immunogenic composition of claim 14, further comprising an adjuvant.

16. A method of eliciting an immune response against an influenza antigenic epitope in a subject, comprising administering to the subject the immunogenic conjugate of claim 1, thereby eliciting an immune response in the subject.

17. The method of claim 16, wherein the immune response is elicited against an influenza HA protein.

18. A method of treating, inhibiting, and/or preventing an influenza infection in a subject, comprising:
selecting a subject for treatment that has, or is at risk for developing, an influenza infection; and
administering to a subject a therapeutically effective amount of the immunogenic conjugate of claim 1, thereby treating, inhibiting, and/or preventing the influenza infection in a subject.

19. (canceled)

20. The method of claim 16, wherein the subject is a human subject.

21-23. (canceled)

24. The method of claim 18, wherein the subject is a human subject.
